(19) United States
(12) Patent Application Publication
(10) Pub. No.: US 2022/0117488 A1
(43) Pub. Date: Apr. 21, 2022

(54) Hermetic Heterogeneous Integration Platform for Active and Passive Electronic Components

(71) Applicant: InjectSense, Inc., Emeryville, CA (US)

(72) Inventors: Jose Padovani, Emeryville, CA (US); Tamir Moran, Emeryville, CA (US); Ates Gurcan, Emeryville, CA (US); Cenk Acar, Emeryville, CA (US); David Marx, Emeryville, CA (US); Ariel Cao, Emeryville, CA (US)

(73) Assignee: InjectSense, Inc., Emeryville, CA (US)

(21) Appl. No.: 17/566,930

(22) Filed: Dec. 31, 2021

Related U.S. Application Data
(63) Continuation of application No. PCT/US2020/040759, filed on Jul. 2, 2020.
(60) Provisional application No. 62/870,243, filed on Jul. 3, 2019.

(51) Int. Cl. A61B 3/16; H05K 5/06

(52) U.S. Cl. CPC A61B 3/16; H05K 5/069; A61B 2562/12; A61B 2562/16; A61B 2562/0247

(57) ABSTRACT

A platform for hermetic heterogeneous integration of passive and active electronic components is provided herein. The platform can include a substrate that provides a hermetic electrical interconnection between integrated circuits and passive devices, such as resistors, capacitors, and inductors. Such substrates can be formed of a dielectric, such as a ceramic, and include electrical interconnects and can further include one or more passive devices. The substrate can include one or more cavities, at least a primary cavity dimensioned to receive an active device and one or more secondary cavities can be included for secondary connector pads for interfacing with the active and passive devices and which can be separately hermetically sealed. The substrate can include a multi-coil inductor defined within alternating layers of the substrate within sidewalls that surround the primary cavity to minimize size of the device package while optimizing the size of the coil.

### CROSS-REFERENCES TO RELATED APPLICATIONS

The present application is a Continuation of PCT Appln No. PCT/US2020/040759 filed Jul. 2, 2020; which claims the benefit of U.S. Provisional Appln No. 62/870,243 filed Jul. 3, 2019, the disclosures which are incorporated herein by reference in their entirety for all purposes.

The present application is generally related to the following co-assigned applications: U.S. Provisional Patent Appln Nos. 62/019,826 filed Jul. 1, 2014, entitled “Methods and Devices for Implantation of Intraocular Pressure Sensors”; 62/019,841 filed Jul. 1, 2014, entitled “Hermetically Sealed Implant Sensors with Vertical Stacking Architecture”; and 62/044,895 filed Sep. 2, 2014, entitled “Ultra Low Power Charging Implant Sensors with Wireless Interface for Patient Monitoring”; the disclosures which are incorporated herein by reference in their entirety for all purposes.

### FIELD OF THE INVENTION

The invention pertains to a multipurpose substrate for hermetic heterogeneous integration of passive and active electronic components, in particular, a substrate that provides hermetic interconnection between integrated circuits and passive or active devices, such as resistors, capacitors, inductors, diodes, transistors or other integrated circuits.

### BACKGROUND OF THE INVENTION

Standard hermetic integration methods for passive and active electronic components use titanium or ceramic cans. These solutions are widely used in applications where the electronic components must be isolated from the external environment such as inside the human body or other aqueous environments. The main advantage of this method is the robustness of the package and the well-established large-volume manufacturing. The main disadvantages are the relatively large size, and limited coupling between external environment and sensors inside the can.

Still other aspects of conventional designs present challenges to further integration and reduction in size. FIGS. 2A-2C depict conventional prior art hermetic designs of inductor coils and implantable sensor chip packages. FIG. 2A shows a conventional design of a hermetic inductor coil 200 having an inductor coil 201 that substantially occupies the available space. Such an inductor would conventionally be surface mounted on a component and would require hermetic encapsulation to seal the electrical connection between the inductor and component. FIG. 2B illustrates a conventional implantable device 210 having a conventional inductor coil layer 211 disposed atop an underlying planar device. Similar to the design in FIG. 2A, the coil substantially occupies the available space and the coil increases the size and/or thickness of the overall device. Reducing the size and space the coil occupies is challenging since it is generally desirable for the inductor coil to be of a relatively large diameter so as to increase the range at which the inductor coil can receive energy for communication or powering of the attached device. FIG. 2C illustrates a conventional die package 220. As can be seen, the package includes a relatively small chip device 221 with wire-bonds 222 to connector pads on a larger epoxy-based package 223 to allow testing/programming of the chip device. The use of wire bonds substantially increases the overall size of the package. To use such a package in an implantable application would require encapsulating the entire chip package within a hermetic seal, such as in a hermetically sealed can, so as to be suitable for implantation. Thus, these approaches are not amenable to reducing the size of the device while still providing hermeticity.

Therefore, there exists a need for an approach that allows for improved integration of passive and electronic components that provides hermeticity and allows further reduction in size, particularly for implantable medical device applications.

### BRIEF SUMMARY

In one aspect, the invention pertains to a hermetic integration platform for active and passive devices. The platform can include a substrate formed of a dielectric material and having electrical interconnects embedded and hermetically sealed within; multiple electrical connection pads disposed on a first portion of a planar surface of the substrate and electrically connected to the interconnects; multiple test pads disposed on a second portion of the substrate and electrically connected to the electrical connection pads through the interconnects; and a seal ring surrounding the electrical connection pads within the first portion so as to facilitate hermetic sealing of the electrical connection pads when electrically coupled and bonded to an active device. The hermetic integration platform can further include one or more passive or active electrical components embedded within and electrically connected to the electrical interconnects. The electrical component can include one or more of a resistor, a capacitor, an inductor, a diode, a transistor, an integrated circuit or any combination thereof.

In some embodiments, the hermetic integration platform includes an inductor coil extending about the perimeter of the substrate. The inductor coil can include multiple coils defined within multiple coil layers alternating with layers of dielectric. The alternating layers can be defined within a sidewall extending about the perimeter so as to define at least a primary cavity dimensioned to receive the active device. In such embodiments, typically, the electrical connection pads and surrounding seal ring are disposed along a bottom surface within the primary cavity to facilitate hermetic and electrical connection of the active device within the primary cavity. The multiple coil layers can be electrically connected by through vias. In some embodiments, the inductor coil further comprises multiple turns within each coil layer defined by alternating lines of a conductive material and a dielectric material, for example, the inductor coil can include eight coils defined along eight layers with three concentric turns within each coil layer. A source pad and a return pad electrically connected to opposite ends of the inductor coil can be included on the second portion of the substrate. It is appreciated that these various features and concepts can be used in a multi-cavity substrate or a single cavity substrate configuration.

In some embodiments, the hermetic integration platform includes a multi-cavity substrate that includes at least the primary cavity and a second cavity separated from the primary cavity by an interior wall, the first portion of the substrate being within the primary cavity and the second portion being within the second cavity. The second cavity can be hermetically sealed with a lid. Typically, the lid is set and sealed permanently, however, in some embodiments, the lid may be removable and/or reusable. In some embodiments, the primary cavity remains open so as to facilitate fluid flow from a surrounding environment around the active device to facilitate a measurement of the environment.

In another aspect, the invention pertains to an implantable sensor that includes a hermetic integration platform, such as described above, and a chip-scale packaged active device having multiple vertically stacked layers so as to form a hermetic seal, for example as described in U.S. Patent Application No. 62/019,841. The chip-scale packaged active device is bonded with the integration platform via the electrical connection pads and the seal ring. In some embodiments, the chip-scale packaged device comprises an integrated circuit (IC) and a MEMS device. The MEMS device can include a pressure sensing device, such as an intraocular pressure (TOP) sensor.

In yet another aspect, the invention pertains to an implant configured to measure an attribute of an environment in which it is implanted. The implant can include an implantable sensor with hermetic integration platform, such as that described above, and a carrier having an open cavity along an intermediate and/or distal portion that is dimensioned for receiving the implantable sensor. In some embodiments, the carrier comprises a sharpened distal end and one or more anchor features at or near a proximal end thereof. In some embodiments, the sensor is a pressure sensor configured for measurement of TOP and the carrier is configured for implantation in an eye of a patient.

In still another aspect, the invention pertains to a method of fabricating a hermetic integration platform for an active device. The method can include steps of: forming a substrate formed of a dielectric material with embedded electrical interconnects so as to be hermetically sealed within; forming electrical connection pads disposed on a first portion of a planar surface of the substrate that are electrically connected to the interconnects; forming a test pads disposed on a second portion of the substrate and electrically connected to the electrical connection pads through the interconnects; and depositing a seal ring surrounding the electrical connection pads within the first portion to facilitate hermetic sealing of the electrical connection pads concurrent with electrical coupling when bonded to an active device. The pads and seal ring may be formed by any suitable deposition method (e.g. printing, electroplating, sputtering, evaporation). Typically, the substrate is a formed of a ceramic (e.g. alumina), although any suitable dielectric material can be used (e.g. high resistivity silicon, silicon dioxide). The interconnects, pads are formed of any suitable conductive material (e.g. gold, platinum). The seal is formed of any suitable material (e.g. gold, gold-indium). In some embodiments, the method includes defining one or more passive electrical components embedded within the substrate during formation thereof such that the passive electrical components are electrically connected via the electrical interconnects and hermetically sealed within. The passive electrical component comprises one or more of a resistor, a capacitor, an inductor, or any combination thereof. The passive component can include an inductor coil formed by alternating coil layers with layers of dielectric, which can extend within a sidewall along a perimeter of the substrate forming an interior cavity for placement of one or more active devices. The interior cavity can further include multiple cavities such as the primary cavity and secondary cavity, as described above.

In yet another aspect, the invention pertains to a method of assembling a device. The method can include steps of: electrically coupling electrical connection contacts of an active device with corresponding electrical connection pads on a substrate formed of a dielectric material, the electrical connection pads being electrically connected to electrical interconnects embedded within the substrate so as to be hermetically sealed; and hermetically sealing the electrical connections between the electrical contacts of the active device and the electrical connection pads of the substrate by bonding a seal ring surrounding the electrical connection pads, the hermetically sealing being concurrent with the electrical coupling. In some embodiments, the electrical coupling and hermetic sealing are performed by thermal compression. The electrical connection pads and seal ring can be planarized or coined before being bonded by thermal compression. In some embodiments, the electrical connection pads are disposed within a primary cavity of the substrate and the embedded interconnects are electrically connected to test pads disposed within a second cavity of the substrate. The method can further include: testing, trimming and/or programming the active device with the test pads after electrically coupling and hermetically sealing the active device within the primary cavity. The method can also include: testing or trimming one or more passive electrical components embedded within and hermetically sealed within the substrate and electrically connected with the test pads in the second cavity, or one or more passive or active devices disposed within the second cavity.

Other features and advantages of the invention shall be apparent based upon the accompanying description, drawings, and claims.

### BRIEF DESCRIPTION OF THE DRAWINGS

FIGS. 1A-1B depicts implantable sensor devices, each having a sensor device within a hermetic integrated substrate with integrated coil in a carrier, in accordance with some embodiments.

FIGS. 2A-2C depict conventional hermetic inductor coils and chip packages.

FIGS. 3-6 depict assembly of an active device within a hermetic integrated multi-cavity substrate with integrated passive devices, in accordance with some embodiments.

FIGS. 7A-7C depict views of an electrical connector of the integrated substrate when bonded with the active device, in accordance with some embodiments.

FIGS. 8A-8B depict additional views of the integrated inductor coil within the substrate, in accordance with some embodiments.

FIGS. 9-12 depict assembly of a device having an active device within a hermetic integrated single-cavity substrate, in accordance with some embodiments.

FIGS. 13A-13B depict views of an electrical connector of the integrated single-cavity substrate when bonded with the active device, in accordance with some embodiments.

FIG. 14 depicts a method of fabrication of a hermetic integrated substrate, in accordance with some embodiments.

FIG. 15 depicts a method of assembly of an implantable device with a hermetic integrated substrate, in accordance with some embodiments.

### DETAILED DESCRIPTION

Although this disclosure is sufficiently detailed to enable one of skill in the art to practice the invention, the embodiments herein disclose mere examples and may be embodied by varying approaches without departing from the scope and spirit of the invention.

In one aspect, the invention overcomes the drawbacks associated with the conventional approaches described above by utilizing an integration platform that integrates hermetic sealing with electrical coupling of electrical components. The substrate can include an integrated electrical component such that the assembly/bonding process concurrently creates a connection between external pads of an electrical device (e.g. IC/MEMS die) and embedded or encapsulated electrical components, and a hermetic seal of the connections. In some embodiments, the invention provides a substrate having a sealing ring disposed about one or more electrical connection pads to facilitate concurrent electrical coupling and hermetic sealing when the substrate is bonded to an electrical device. In some embodiments, the substrate further includes a one or more passive electrical components embedded within the substrate so as to be hermetically sealed. The electrical device can be a chip-scale packaged device having vertically stacked layers that form a hermetic seal such that when coupled to the hermetic integration substrate, the assembly is hermetically sealed without requiring additional hermetic encapsulation of the assembly, such as within a can.

In another aspect, the invention pertains to an integration substrate that integrates heterogeneous materials to facilitate integration of hermetic sealing with electrical coupling of electrical components. In some embodiments, the integration substrate includes a dielectric material (e.g. alumina/ceramic) with alternating layers of a conductor (e.g. gold, platinum) and a dielectric material bonded to form a hermetic seal. In some embodiments, the alternating layers of conductor and dielectric form an electrical component that is hermetically sealed within the substrate and electrically connected to a connection pad in a discrete portion of the substrate. This allows for electrical coupling about a reduced area portion (e.g. less than half the area of a major face) such that a relatively small seal ring can provide hermetic sealing. This approach is also advantageous in that the area that is mechanically coupled is also reduced, which reduces the mechanical stresses on the electrical component. This can reduce or obviate the need to utilize additional mechanical couplings (e.g. posts) to isolate sensitive components from mechanical stresses, as compared to conventional designs where the entire electrical component is mechanically coupled along a majority of the surface. In another aspect, this approach allows a hermetically constructed electrical component to be hermetically, electrically and mechanically coupled to the integration substrate at a first discrete location (e.g. first area), while the substrate includes additional electrical features (e.g. testing pads, passive components, electrical components, such as inductors, resistors) along other areas (e.g. second area). This approach allows the assembly to be hermetically sealed, thereby obviating the need for additional hermetic sealing (e.g. encapsulation, can).

In yet another aspect, the heterogeneous integration substrate provides a housing for hermetically packaged dies and interconnection to passive or active external components that can be either fabricated as part of the substrate (e.g., embedded) or can be discrete components located on a secondary hermetically sealed cavity. The heterogeneous substrate can be configured to provide access to test pads located in one or more secondary cavities and routed to the main die through embedded traces such that the connections are hermetically sealed.

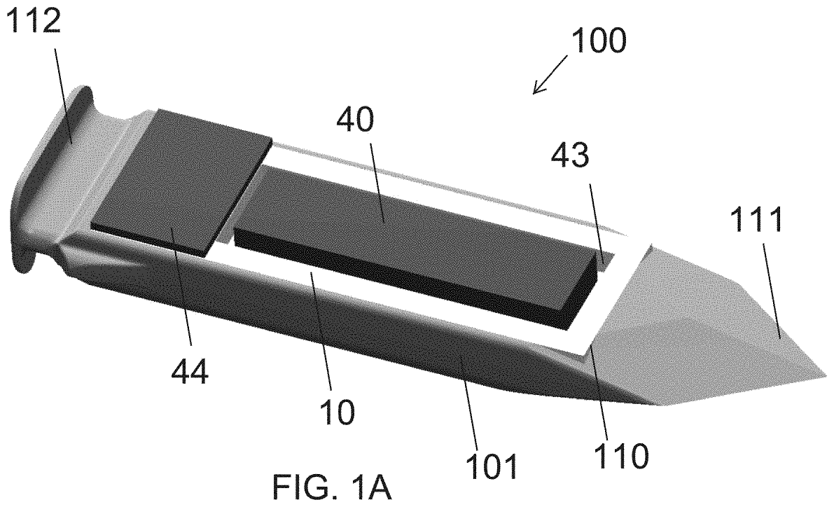

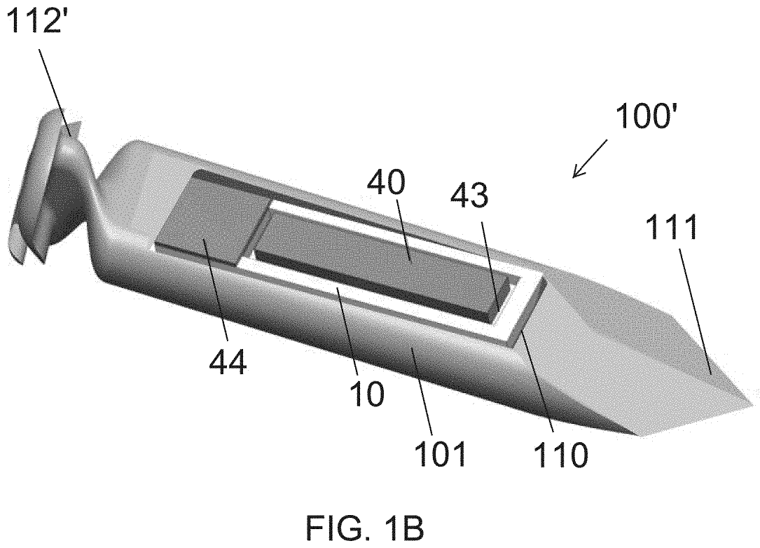

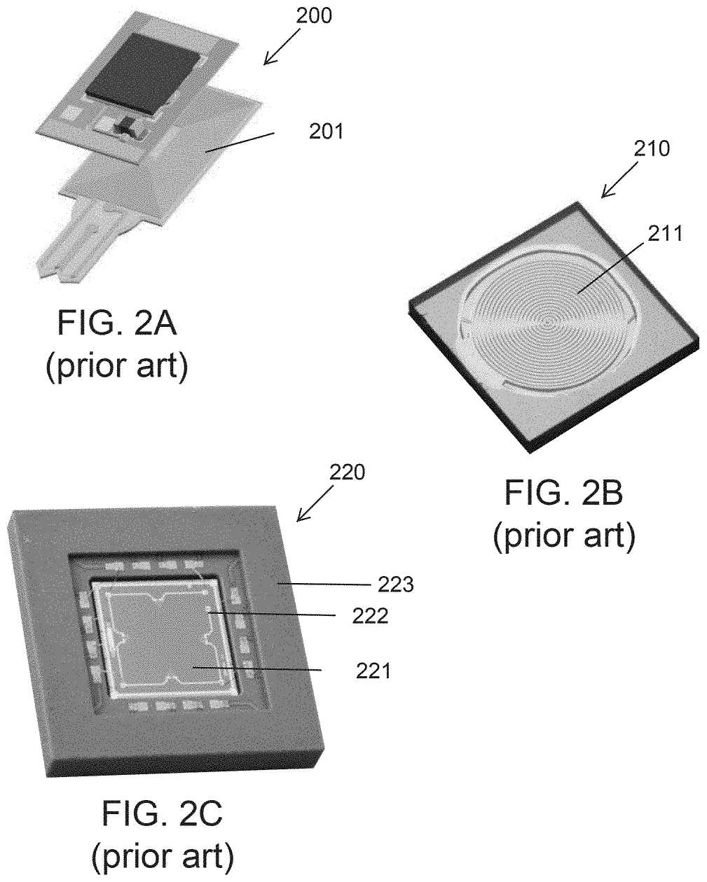

The above described approaches provide various advantages over conventional approaches. For example, hermetically packaged IC/MEMS die can be exposed to the media while still establishing a hermetic connection to other external components. A smaller form factor can be achieved due to the integration for embedded passive components and surface mount components. Surface mount components can be hermetically sealed, for example, by placing non-hermetic surface mount components within the secondary cavity and hermetically sealing the secondary cavity with a lid. Passive components can be hermetically embedded within a substrate. Access to test points can be provided in a secondary cavity, while the active device remains hermetically sealed. It is appreciated that the integration substrate approach may utilize any one or combination of the above features described herein, which can be further understood by referring to the following examples in FIGS. 1 and 3-8B.

FIG. 1A illustrates an example of an implantable sensor device 100 including implantable sensor 40 assembled within a hermetic integration platform and disposed within carrier 101. Since the implantable sensor assembly is already hermetic sealed by virtue of the hermetic integration platform, the sensor is placed within an open cavity 110 of the carrier 101 and is not required to further include a can or additional hermetic encapsulation layers. The implantable sensor is securely coupled to the carrier 101 (e.g. by bonding) before insertion/implantation into the body. In this embodiment, the distal portion of the carrier include a sharped distal tip 111 to facilitate insertion into the body, while a proximal portion of the carrier includes one or more anchor features 112 so as to maintain the position of the implantable sensor within a target area, such as within the eye. The sensor device portion includes an IC/MEMS sensor device 40, having a vertically stacked chip-scale packaging construction, that is disposed in a primary cavity in integration substrate 10, which includes electrical connections to the sensor device 40. In this embodiment, the electrical connections are coupled with test pads disposed within a secondary cavity that is separately hermetically sealed with lid 44. The integration substrate further includes an inductor coil built in layers within the sidewalls of the substrate, which hermetically seals the inductor coil and allows the coil to be suitably sized without increasing the thickness overall sensor device. FIG. 1B illustrates another design of implantable sensor device 100′ having same or similar features as that in FIG. 1A except the proximal anchor features 112′ are configured to be deployed by rotation (e.g. twisting), whereas the anchor features 112 in FIG. 1A can be deployed by pressing longitudinally.

In some embodiments, multiple IOP measurements are obtained from an implantable sensor implanted within the eye. Preferably, the sensor is implanted so that a sensor portion is implanted entirely within the vitreous body so as to obtain improved measurements of IOP, for example by the implantation approach described in U.S. Patent Application No. 62/019,826, which is incorporated herein by reference, or any suitable implantation approach. Such a sensor can include any of the configurations depicted in U.S. Patent Application No. 62/019,841, which is incorporated herein by reference, or any suitable sensor. Further, it is desirable for the IOP sensor to obtain high frequency sampling (e.g. from minutes to hour sampling) and facilitate telemetry of the obtained measurements, for example by any of the approaches described in U.S. Patent Application No. 62/044,895, which is incorporated herein by reference, or any suitable approach.

While the hermetic integration platform is described above with respect to a pressure sensor for implantation into the eye, it is understood that these same concepts could be applied to any types of MEMS device for implantation into any media, including any area of the body or non-biological environments as well. In alternative embodiments, the device may not require a carrier or may utilize a substantially different configuration suited for the particular application.

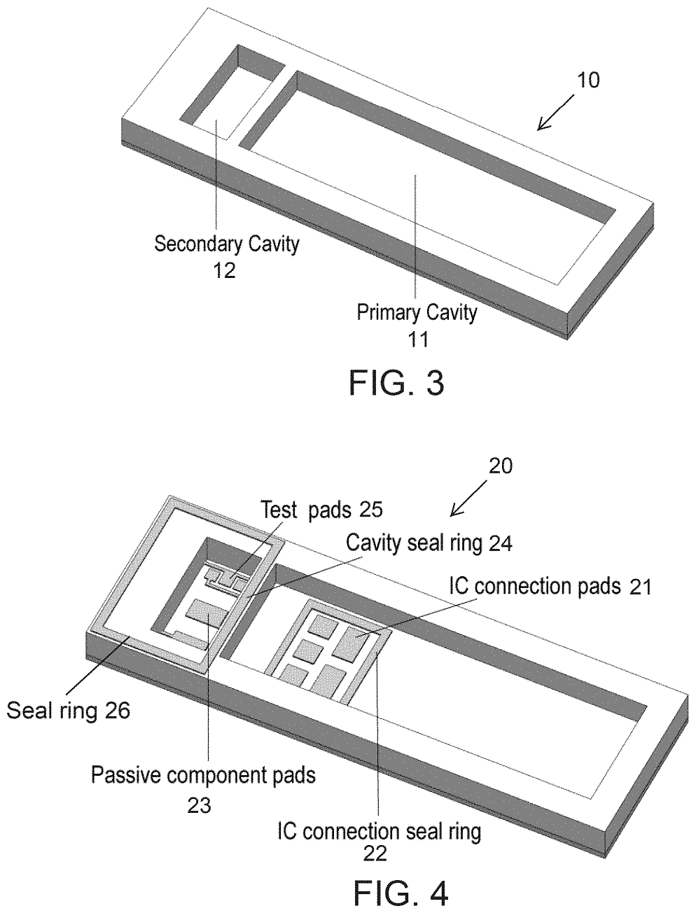

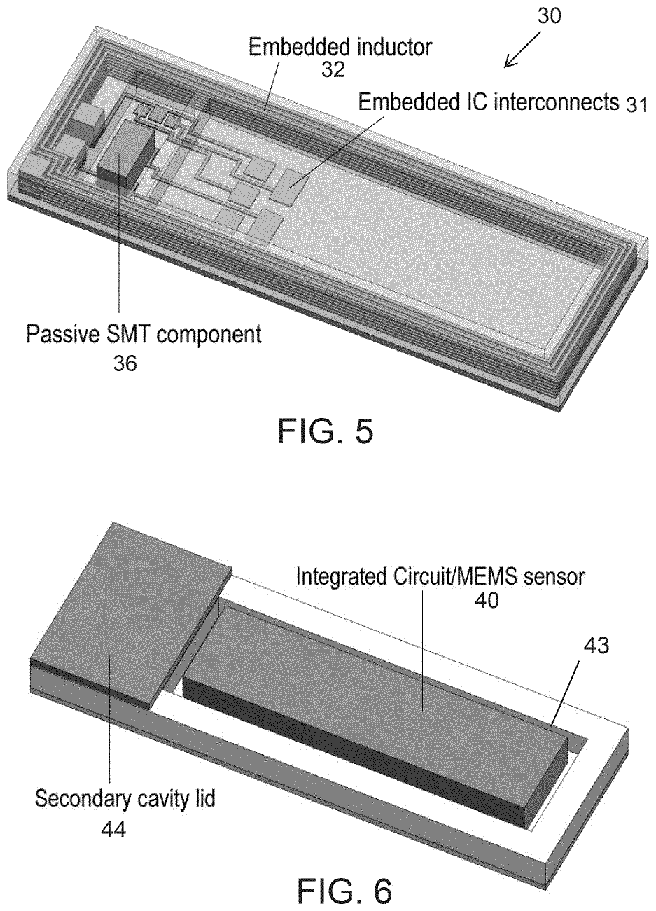

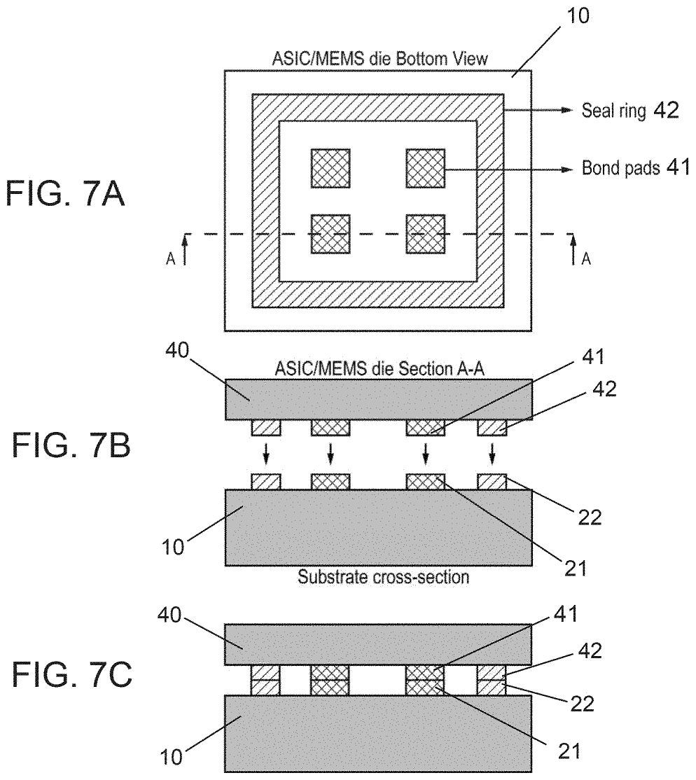

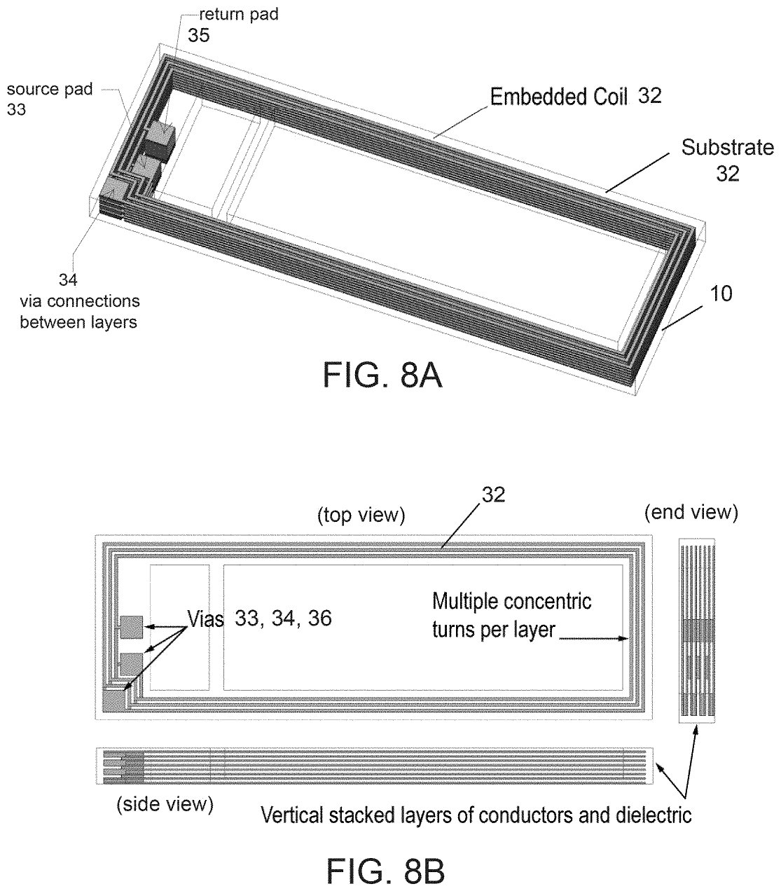

FIGS. 3-8B illustrate the various components of a heterogeneous integration substrate for coupling with an integrated circuit (IC)/MEMS sensor device, in accordance with some embodiments. FIG. 3 illustrates the integration substrate 10 with primary and secondary cavities. FIG. 4 illustrates the electrical connections 20 between connection pads in the primary and secondary cavities. FIG. 5 illustrates the embedded features 30 including the IC interconnects 31 and inductor coil 32 embedded within the substrate and a passive component 36 disposed within the secondary cavity. FIG. 6 illustrates the integrated substrate with the IC/MEMS sensor device 40 disposed within the primary cavity and the secondary cavity hermetically sealed within a lid. FIG. 7A-7C shows the seal ring disposed around the bond pads along the bottom surface of the integration substrate. FIG. 8A-8B show additional details of the embedded inductor coil. Each of the above aspects is discussed in further detail as follows.

FIG. 3 illustrates the heterogeneous integration substrate 10 with multiple cavities that are laterally disposed. Cavity dimensions are determined by the die or the components to be located inside each cavity. The number of cavities is dependent upon the application and can vary from a single cavity to multiple cavities. In this embodiment, the substrate includes a primary cavity 11 and secondary cavity 12. Primary cavity 11 is sized and dimensioned for housing the IC/MEMs device 40 (shown in FIG. 6). The cavity depth can be such that the die is recessed or protruding from the cavity. The secondary cavity is separated by substrate walls, which can be formed integrally with the perimeter sidewall or formed subsequent to formation of the perimeter sidewall. While the secondary cavity is shown here as being smaller than the primary cavity, it is understood that the secondary cavity could be the same size or larger than the primary cavity. Further, in some embodiments, the substrate could include one or more additional cavities.

FIG. 4 shows the electrical connections 20 within the integrated substrate 10. The primary cavity includes IC connection pads 21 on the bottom surface and a seal ring 22 around the connection pads. The IC connection pads 21 are arranged so as to couple with corresponding connection pads of the IC/MEMS die when bonded within the primary cavity. Seal ring 22 hermetically seals the connection and isolates the connection pads 21 from external media. Secondary cavity can include passive component pads 23 for surface mounted (SMT) passive or active electronic components or other ICs and provide test access pads 25, which are electrically coupled with the connection pads 21 within the primary cavity. The test pads 25 can be used to test the IC/MEMS device once bonded within the primary cavity. The top surface of the secondary cavity includes a seal ring 26 used to hermetically seal the secondary cavity with a lid after testing of the IC/MEMS device is complete.

FIG. 5 shows the integration substrate 10 with embedded components 30 that include electrical interconnects 31 between the primary and secondary cavity and an embedded inductor coil 32. The interconnects 31 are made by embedded traces that are routed between cavities, such as by extending through the substrate under the wall separating the cavities. As shown, interconnects 31 extend between the IC connection pads 21 and connection pads within the secondary cavity. The interconnects 31 are embedded within the substrate during fabrication such that they are hermetically sealed. The interconnects are further electrically connected to the inductor coil 32, which is embedded within the sidewalls of the substrate during fabrication such that the inductor coil 32 is also hermetically sealed. Surface mounted components 36, such as resistors, capacitors, inductors or light emitting diodes (LED), among others, can be integrated within the secondary cavity, or one or more additional cavities.

FIG. 6 shows the integration of sensor device with the IC/MEMS sensor device 40 disposed within the primary cavity with the secondary cavity hermetically sealed with lid 44. While the secondary cavity is shown as being hermetically sealed with a lid, it is appreciated that in other embodiments, both cavities could be hermetically sealed with a lid depending on the type of MEMS device and application. In some embodiments, the sensor device may include an optical device (e.g. LED, photodetector) within the primary or secondary cavity. In such cases, the lid may be optically transparent (e.g. glass). As shown, a gap 43 extends about the IC/MEMS sensor 40 such that fluid from the environment can flow into the gap and beneath the IC/MEMS sensor, which provides more accurate sensor measurements of the environment. In this embodiment, the IC/MEMS sensor device is mechanically coupled near a proximal end thereof by the seal ring (see FIG. 4) such that the distal portion of the sensor device, which includes the sensing portion, is cantilevered. This approach further isolates the sensing portion from mechanical stresses and deformation applied through the substrate so a so provide more accurate sensor measurements (e.g. measurements of IOP). It is appreciated, however, that the design could be mechanically coupled elsewhere or further include one or more posts or other mechanical coupling feature along an intermediate or distal portion to improve mechanical coupling if needed.

FIG. 7A-7C further illustrate the bonding process of the connection pads 21 and the seal ring 22 of the integration substrate 10 to the IC/MEMS sensor device 40. In this embodiment, the connection pads 21 and the seal ring 22 are fabricated at the bottom of the primary cavity and with the same material and with the same thickness to enable mechanical bonding to a corresponding geometry of connectors on the bottom surface of the IC/MEMS device 40. FIG. 7A shows a bottom view of die depicting connection pads 41, for electrical interconnects to passive components and/or test pads, and a seal ring 42 for hermetic isolation of bond pads from external media. FIG. 7B illustrates a cross sectional view of bottom connectors 41 and seal ring 42 on device die 40 that correspond to pads 21 and seal ring 22 on the top surface of the substrate at the bonding locations, which show a matching pattern on both sides. Typically, both bond pads and seal ring have same height and same material to facilitate successful simultaneous bonding. FIG. 7C illustrates a cross sectional view of the contact portion of device die 40 and substrate after bonding. In some embodiments, the connection pads and seal ring are formed of a conductive paste (e.g. gold, platinum) and printed on the connector portion and are bonded with the pads of the device by thermal compression. In some embodiments, the connector layer is planarized or coined prior to thermal compression.

FIGS. 8A-8B further illustrate the passive inductor coil 32 integrated within the walls of the substrate 10 along the perimeter of the cavity 13. This approach allows the coil to be of a larger area than that of the chip device disposed within cavity 13 to which the coil is electrically connected. This increases the depth at which the inductor coil can operated. The inductor coil can serve as a wireless communication and powering device by means of the mutual inductance method. In this embodiment, the inductor coil is formed by depositing alternating coil layers having a line of a conductor (e.g. gold) with layers of an insulator (e.g. dielectric, ceramic). Each coil layer can include multiple lines of conductor alternating with dielectric to form a multi-turn coil. Vertical via connections 34 are used to connect the conductor layers, as can be seen in the side view and end view shown in FIG. 8B. Concentric turns of conductor lines are used to increase the number of turns per layer and the serial connection of vertical layers gives the total number of turns. In this embodiment, the embedded coil has three concentric turns per layer with eight total stacked conductor layers, for a total of twenty-four turns. Typically, each layer has thicknesses on the order of 10-20 micrometers and the overall height of the substrate is determined by the number of stacked layers.

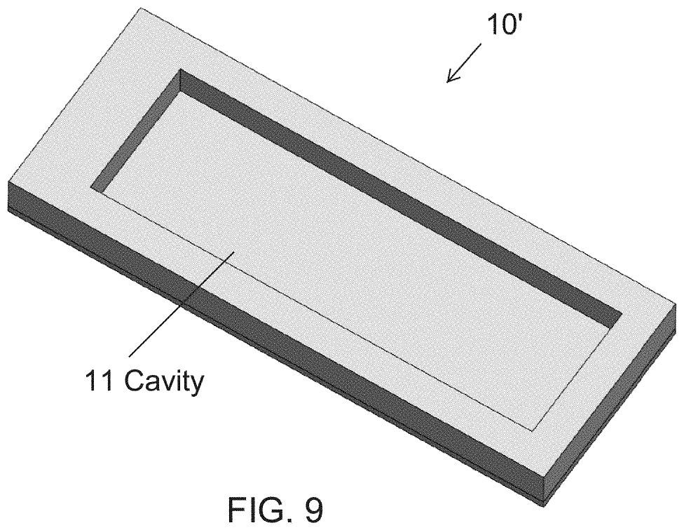

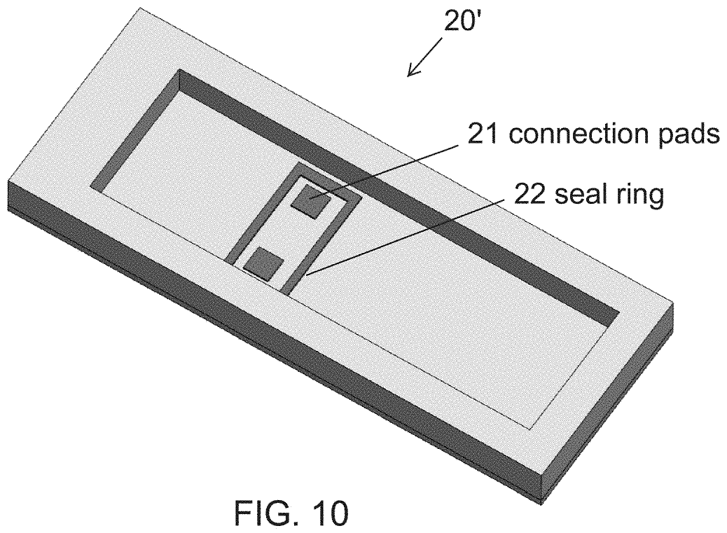

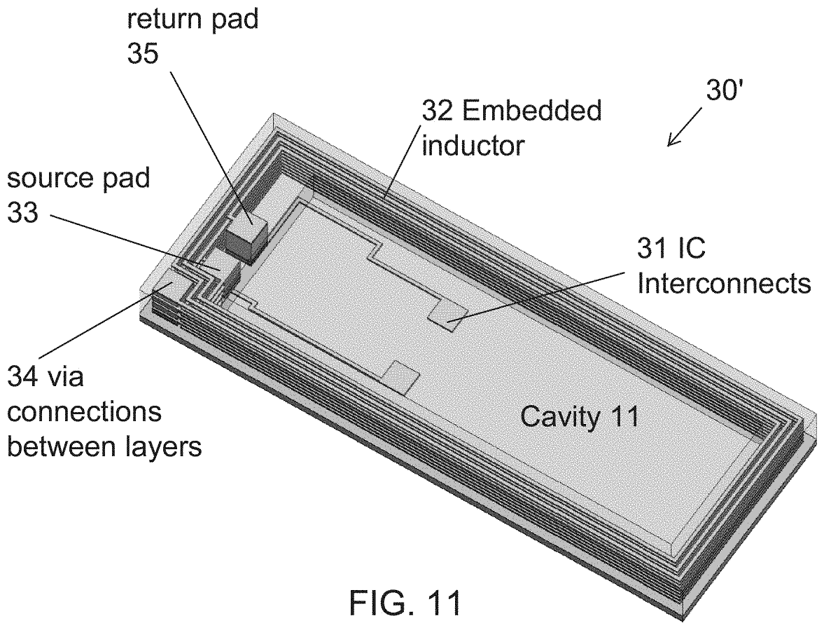

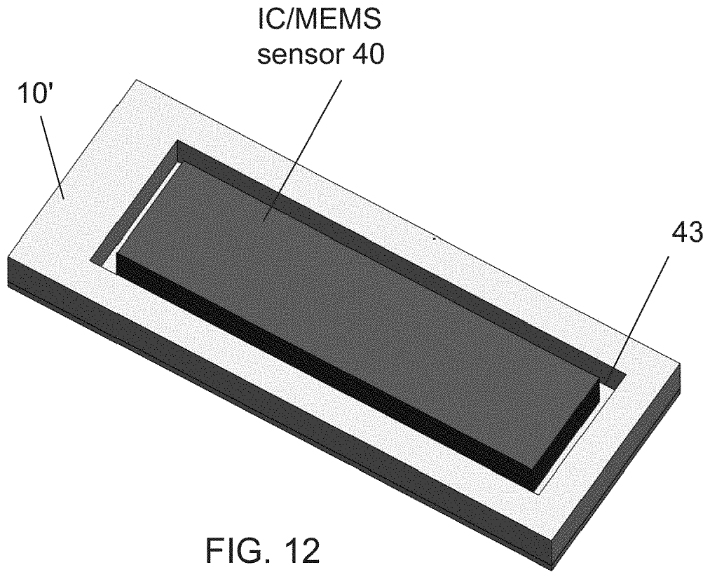

FIGS. 9-12 illustrate the various components of a heterogeneous integration single-cavity substrate with integrated passive device for coupling with a device die (e.g. integrated circuit IC/MEMS sensor device), in accordance with some embodiments. These components include same or similar features as the integration substrate in FIG. 3-6 except without the secondary cavity having additional electrical pads for testing. It is appreciated that the various concepts and assembly steps described previously can be applied to a single-cavity integrated substrate as well. FIG. 9 illustrates the integration substrate 10′ with a single cavity 11. FIG. 10 illustrates the electrical connections 20′ that include connection pads 21 in the single cavity and a seal ring 22 around the connection pads. FIG. 11 illustrates the embedded features 30′ including the IC interconnects 31 and inductor coil 32 embedded in the sidewalls of the substrate. As described previously, the inductor coil 32 is defined by multiple layers of conductors that are electrically connected by via connections 34 and connected to the IC interconnects by source pad 33 and return pad 35. FIG. 12 illustrates the integrated substrate 10′ with the IC/MEMS sensor device 40 disposed within the single cavity, there being a gap 43 surrounding the IC/MEMS sensor device 40.

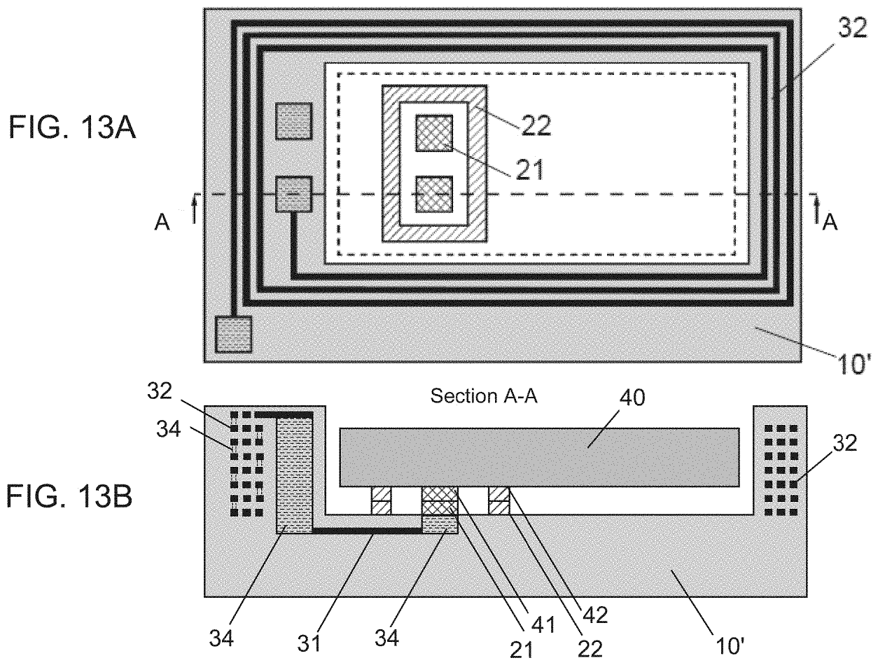

FIGS. 13A-13B shows an overview and cross-sectional side view, respectively, of the IC/MEMS sensor device 40 connected and sealed by the seal ring disposed around the bond pads along the bottom surface of the integration substrate 10′. In FIG. 13A, the outline of the die device 40 is indicated in dashed line. Similar to the embodiment in FIGS. 3-6, the electrical contact pads 41 and seal ring 42 of the device 40 are bonded to the corresponding electrical contact pads 21 and seal ring 22 of the integration substrate 10′, which connects the device 40 to the inductor coil 42 embedded in the sidewalls of the substrate 10′. In FIG. 13B, the embedded IC interconnects 31 connect the electrical pad 21 to the coils of the inductor coil 32 through vias 34.

As can be seen in FIG. 13B, the gap 43 extends around and beneath the IC/MEMS sensor device 40 because a substantial portion of the device is cantilevered from the seal ring support portion. This cantilever aspect also holds true of the embodiment in FIG. 6. This configuration allows flow of fluid around the sensing portion of the IC/MEMS sensor device and isolates the sensing portion from mechanical stresses and deformation, thereby providing more accurate pressure measurements.

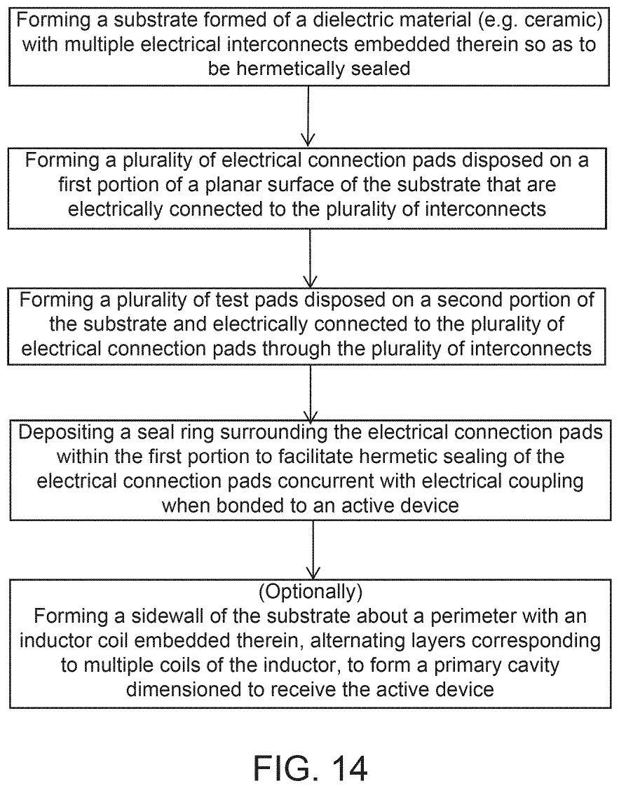

FIG. 14 depicts a method of fabricating a hermetic integration substrate for active device and passive devices. The method includes steps of: forming a substrate formed of a dielectric material (e.g. ceramic, such as alumina) with multiple electrical interconnects (e.g. traces) embedded therein so as to be hermetically sealed; forming electrical connection pads disposed on a first portion of a planar surface of the substrate that are electrically connected to the interconnects; and forming test pads disposed on a second portion of the substrate and electrically connected to the electrical connection pads through the interconnects. The traces and pads can be formed of any suitable conductor (e.g. gold, platinum). The method further includes depositing a seal ring surrounding the electrical connection contacts within the first portion to facilitate hermetically sealing of the electrical connection pads concurrent with electrical coupling when bonding the active device. The method can further include forming a sidewall of the substrate about a perimeter with an integrated inductor coil embedded therein, alternating coil layers corresponding to multiple coils of the inductor coil. The sidewall can be defined so as to form a primary cavity within that is dimensioned to receive the active device. The method can further include integration of one or more passive or active devices (e.g. resistor, capacitor, inductor, diodes, transistors, other ICs) within the substrate or within a secondary cavity.

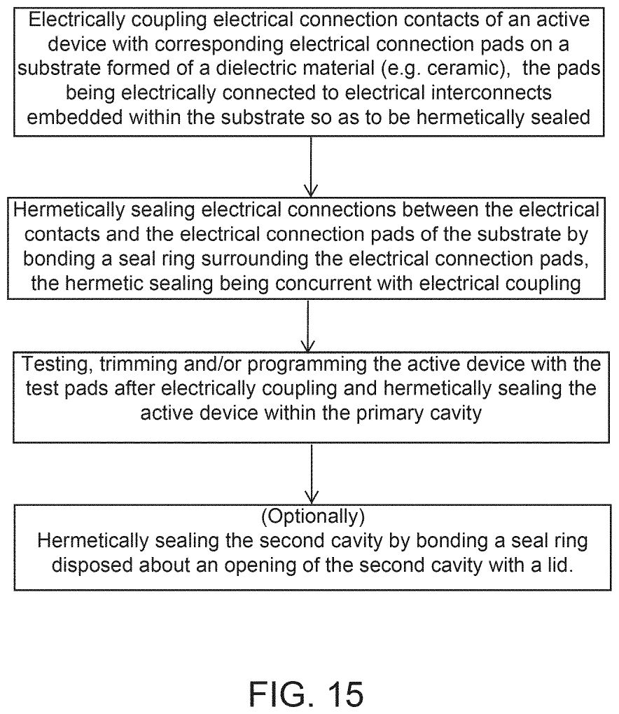

FIG. 15 depicts a method of assembling an active device with a hermetic integration substrate. The method includes steps of: electrically coupling electrical connection contacts of an active device with corresponding electrical connection pads on a substrate formed of a dielectric material (e.g. ceramic), the pads being electrically connected to electrical interconnects embedded within the substrate so as to be hermetically sealed; hermetically sealing electrical connections between the electrical contacts and the electrical connection pads of the substrate by bonding a seal ring surrounding the electrical connection pads, the hermetically sealing being concurrent or simultaneous with electrical coupling. The method can further include testing, trimming and/or programming the active device (as well as any other passive devices integrated within the substrate or in a secondary cavity) with the test pads or other pads disposed in the second cavity. Any secondary cavities can then be hermetically sealed by bonding a seal ring disposed about an opening of the respective cavity with a lid.

It is appreciated that any of the above concepts can be used as described above, individually or in combination, or can be modified according to various alternatives. Such alternatives include use of different dielectric materials and/or fabrication methods (e.g. multilayer thick film process, low-temperature co-fired ceramic (LTCC), 3D printing/scaffolding, silicon). In some embodiments, the design could include a single cavity or could include multiple cavities for both active and passive electronic components. Cavity dimensions can vary depending on the size of the components or as needed for a particular application. In some embodiments, one or more cavities can be sealed with a lid, left open or multiple cavities could be sealed with single lid. In some embodiments, a cavity can be sealed with a lid of a material that is optically transparent, RF transparent or opaque.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

1. A hermetic integration platform comprising:
a substrate formed of a dielectric material and having a plurality of electrical interconnects embedded and hermetically sealed therein;
a first plurality of electrical connection pads disposed on a first portion of a planar surface of the substrate and electrically connected to the plurality of interconnects;
a second plurality of electrical connection pads disposed on a second portion of the substrate and electrically connected to the first plurality of electrical connection pads on the first portion through the plurality of interconnects; and
a seal ring surrounding at least the plurality of electrical connection pads within the first portion so as to facilitate hermetic sealing of the plurality of electrical connection pads when electrically coupled and bonded to an active device.

2. The hermetic integration platform of claim 1 further comprising:
one or more passive electrical components embedded therein and electrically connected to the electrical interconnects.

3. The hermetic integration platform of claim 2 wherein the passive electrical component comprises one or more of a resistor, a capacitor, an inductor, a diode or any combination thereof.

4. The hermetic integration platform of claim 2 wherein the passive electrical component is an inductor coil extending about the perimeter of the substrate.

5. The hermetic integration platform of claim 4 wherein the inductor coil comprises a plurality of coils defined within a plurality of coil layers alternating with layers of dielectric.

6. The hermetic integration platform of claim 5 wherein the alternating plurality of layers are defined within a sidewall extending about the perimeter so as to define at least a primary cavity dimensioned to receive the active device.

7. The hermetic integration platform of claim 6 wherein the plurality of electrical connection pads and surrounding seal ring are disposed along a bottom surface within the primary cavity to facilitate hermetic and electrical connection of the active device within the primary cavity.

8. The hermetic integration platform of claim 7, wherein the plurality of coil layers are electrically connected by through vias.

9. The hermetic integration platform of claim 8, wherein the coil further comprises a plurality of turns within each coil layer defined by alternating lines of a conductive material and a dielectric material.

10. The hermetic integration platform of claim 9, wherein the coil comprises eight coils defined along eight layers with three concentric turns within each coil layer.

11. The hermetic integration platform of claim 8, further comprising:
a source pad and a return pad electrically connected to opposite ends of the inductor coil, wherein the source pad and the return pad are disposed on the second portion of the substrate.

12. The hermetic integration platform of claim 6 wherein the substrate comprises a second cavity separated from the primary cavity by an interior wall, wherein the second portion is within the second cavity.

13. The hermetic integration platform of claim 12 wherein the second cavity is hermetically sealed with a lid.

14. The hermetic integration platform of claim 13 wherein the primary cavity remains open so as to facilitate fluid flow from a surrounding environment around the active device to facilitate a measurement of the environment.

15. An implantable sensor comprising:
the integration platform of claim 1; and
a chip-scale packaged active device having a plurality of vertically stacked layers so as to form a hermetic seal, wherein the chip-scale packaged active device is bonded with the integration platform via the plurality of electrical connection pads and the seal ring.

16. The implantable sensor of claim 15 wherein the chip-scale packaged device comprises an integrated circuit (IC) and a MEMS device.

17. The implantable sensor of claim 16 wherein the MEMS device comprises a pressure sensing device.

18. An implant comprising:
the implantable sensor of claim 17; and
a carrier having an open cavity along an intermediate portion that is dimensioned for receiving the implantable sensor.

19. The implant of claim 18, wherein the carrier comprises a sharpened distal end and one or more anchor features at or near a proximal end thereof.

20. The implant of claim 19, wherein the sensor is a pressure sensor configured for measurement of intraocular pressure (TOP) and the carrier is configured for implantation in an eye of a patient.

21.-60. (canceled)
